(19) United States
(12) Patent Application Publication
(10) Pub. No.: US 2008/0102497 A1
(43) Pub. Date: May 1, 2008

(54) ENZYMATIC HYDROLYSIS OF STARCH

(76) Inventors: Dominic Wong, El Cerrito, CA (US); Arun Kumar Uppalanchi, Foxboro, MA (US); Xiangdong Gan, North Attleboro, MA (US); Bin Zhong, Cumberland, RI (US)

Correspondence Address: CALFEE HALTER & GRISWOLD, LLP, 800 SUPERIOR AVENUE, SUITE 1400, CLEVELAND, OH, 44114, US

(21) Appl. No.: 11/619,314

(22) Filed: Jan. 3, 2007

Related U.S. Application Data
(60) Provisional application No. 60/855,671, filed on Oct. 31, 2006.

(51) Int. Cl. C12P 19/04; C12P 19/02; C12N 9/32

(52) U.S. Cl. 435/101; 435/204; 435/105

(57) ABSTRACT

Provided herein are methods of increasing the enzymatic rate of hydrolysis of starch substrates. In certain embodiments the method comprises contacting a starch substrate with one or more amylase family enzymes in the presence of greater than 0.001 mM manganese ions. In certain embodiments, the method comprises contacting a starch substrate with one or more glucoamylase and/or β-amylase enzymes in the presence of greater than 0.001 mM calcium ions (Ca⁺⁺). In certain embodiments, the method comprises contacting the starch substrate with a β-amylase in the presence of greater than 0.001 mM manganese ion, calcium ion, magnesium ion (Mg⁺⁺), strontium ion (Sr⁺⁺), barium ion (Ba⁺⁺) or any combination of said metal ions. In certain embodiments the method comprises contacting the starch substrate with a glucoamylase in the presence of greater than 0.001 mM manganese ion, calcium ion, lithium ion (Li⁺), potassium ion (K⁺), or any combination of said metal ions. Also provided herein are compositions and kits for hydrolyzing starch.

This application claims benefit of U.S. Provisional Application No. 60/855,671, filed Oct. 31, 2006, which is incorporated herein by reference in its entirety.

The present application relates to methods of starch hydrolysis by the amylase family of enzymes.

Starch is one of the most common storage carbohydrates found in plants. Plants with high starch content include corn, potato, rice, sorghum, wheat, and cassava. Starch from all plant sources occurs in the form of granules which differ markedly in size and physical characteristics from species to species.

Starch consists of two types of polymers, amylose and amylopectin. Amylose is the linear fraction containing α-1,4 linked glucose homopolymers and a few α-1,6 branch points. Amylopectin is the branched fraction consisting of α-1,4 glucose chains linked to other α-1,4 glucose chains by α-1,6 branch points. The degree of branching in amylopectin is approximately one per twenty glucose units in the unbranched segments. Some starches, for instance from potato, also contain covalently bound phosphate in small amounts (approximately 0.06-0.2%).

The starch produced in each plant consists of a variable percentage of each of its amylose and amylopectin constituents, or even of a particular molecular weight distribution of each of the glucose homopolymers. These differences can affect properties such as the solubility profile, gelling properties, and reactivity of starch from different sources.

The hydrolytic products of starch have commercial application in the agriculture, food, chemical, pharmaceutical, and fuel industries. For example, starch can be hydrolyzed to glucose, which can then be used for producing fuel ethanol. Other areas of application include, but are not limited to, the production of sweeteners such as corn syrup, high fructose syrup, maltose syrup, the production of brewages such as beer alcohol, bakeries, nutraceuticals, pharmaceuticals and medical uses, such as products used to facilitate digestion of starchy foods.

Although acid hydrolysis of starch was commonly used in the past to convert starch to glucose and/or other hydrolytic products, it has largely been replaced by enzymatic processes.

Typically, there are three stages in the enzymatic conversion of starch to glucose and maltose. These include:
1. Gelatinization, involving the dissolution of starch granules to form a viscous suspension;
2. Liquefaction, involving the partial hydrolysis of the starch, with concomitant loss in viscosity; and
3. Saccharification, involving the production of glucose and maltose by further hydrolysis.

Gelatinization is achieved by heating starch with water, and occurs naturally when starch is cooked. Gelatinized starch is readily liquefied by partial hydrolysis with enzymes or acids and saccharified by further acidic or enzymatic hydrolysis

Enzymes that are typically used in starch hydrolysis are shown in Table 1 below:

TABLE 1

| Enzyme | EC number | Source | Action |
|---|---|---|---|
| α-Amylase | 3.2.1.1 | Bacillus amyloliquefaciens | Only α-1,4 linkages are cleaved to |
| | | | give α-dextrins and predominantly |
| | | | maltose (G2), G3, G6 and G7 |
| | | | oligosaccharides |
| | | B. licheniformis | Only α-1,4 linkages are cleaved to |
| | | | give α-dextrins and predominantly |
| | | | maltose, G3, G4 and G5 |
| | | | oligosaccharides |
| | | Aspergillus oryzae, | Only α-1,4 linkages are cleaved to |
| | | A. niger | give α-dextrins and predominantly |
| | | | maltose and G3 oligosaccharides |
| | | B. subtilis (amylosacchariticus) | Only α-1,4 linkages are |
| | | | cleaved to give α-dextrins |
| | | | with maltose, G3, G4 and up |
| | | | to 50% (w/w) glucose |
| β-Amylase | 3.2.1.2 | Malted barley | Only α-1,4 linkages are cleaved, from |
| | | | non-reducing ends, to give limit dextrins |
| | | | and β-maltose |
| Glucoamylase | 3.2.1.3 | A. niger | α-1,4 and α-1,6-linkages are cleaved, |
| | | | from the non-reducing ends, to give β- |
| | | | glucose |

In view of the commercial importance of starch hydrolytic products, there remains a need in the art for methods that increase the rate of enzymatic hydrolysis of substrates containing starch, amylose, and/or amylopectin.

### SUMMARY OF THE INVENTION

Provided herein are methods of improving the rate of enzymatic hydrolysis of starch substrate. In certain embodiments the method comprises contacting a starch substrate with one or more amylase family enzymes in the presence of greater than 0.001 mM manganese ions (Mn⁺⁺), provided that the one or more amylase family enzymes is not the glucoamylase from Neurospora crassa. In certain embodiments, the starch substrate, manganese ion, and enzymes are contacted in a reaction mixture that has a pH of 8 or less and, preferably, comprises a buffer. In certain embodiments, the method comprises contacting a starch substrate with one or more glucoamylase and/or β-amylase enzymes in the presence of greater than 0.001 mM calcium ions (Ca⁺⁺). In certain embodiments, the starch substrate is contacted with one or more amylase family enzymes in the presence of greater than 0.001 mM manganese ions and greater than 0.001 mM calcium ions. In certain embodiments, the glucoamylase is one or more of the following: the glucoamylase from Rhizopus Sp, the glucoamylase from Aspergillus niger, and the glucoamylase purified from mushroom. In certain embodiments, the α-amylase is one or more of the following: the α-amylase from Aspergillus oryzae, pancreatic α-amylase, the α-amylase from Bacillus amyloliquefaciens, the α-amylase from Bacillus licheniformis, the α-amylase from Bacillus subtilis, the α-amylase from human saliva, the α-amylase from barley, the industrial α-amylase from Novozymes known as Termamyl. In certain embodiments, the β-amylase is one or both of the following: the β-amylase from barley and the β-amylase from sweet potato. In certain embodiments, the method comprises contacting the starch substrate with a β-amylase in the presence of greater than 0.001 mM manganese ion, calcium ion, magnesium ion (Mg⁺⁺), strontium ion (Sr⁺⁺), barium ion (Ba⁺⁺) or any combination of said metal ions. In certain embodiments the method comprises contacting the starch substrate with a glucoamylase in the presence of greater than 0.001 mM manganese ion, calcium ion, lithium ion (Li⁺), potassium ion (K⁺), or any combination of said metal ions.

Also provided herein are compositions and kits for hydrolyzing starch. In certain embodiments, the compositions comprise one or more α-amylase enzymes, one or more β-amylase enzymes and/or one or more glucoamylase enzymes and greater than 0.001 mM manganese ion. In certain embodiments, the kit comprises one or more α-amylase enzymes, one or more β-amylase enzymes, and/or one or more glucoamylase enzymes and manganese ion, calcium ion, magnesium ion, strontium ion, barium ion, lithium ion, potassium ion or any combination of said metal ions. The kits also comprises one or more containers. In certain embodiments, the kits also comprises instructions for using the enzymes and ions to hydrolyze a starch substrate.

### BRIEF DESCRIPTION OF THE FIGURES

FIG. 1 is a schematic representation of the steps and enzymes used in one embodiment of starch hydrolysis.

FIG. 2 is a graph showing the pH effect on the relative activity of the glucoamylase from Rhizopus sp on raw corn starch in a reaction mixture with and without manganese ion (Mn⁺⁺).

FIG. 3 is a bar graph showing the temperature effect on the conversion rate of raw corn starch substrate by the glucoamylase from Rhizopus sp in a reaction mixture with and without Mn++.

FIG. 4 is a graph showing the pH effect on the relative activity of the β-amylase from barley on cooked dent corn starch in a reaction mixture with and without Mn⁺⁺.

### DETAILED DESCRIPTION OF THE INVENTION

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurement.

All publications, patent applications, patents, and other references mentioned herein are incorporated by reference in their entirety.

### Definitions.

The term “amylase family of enzymes” as used herein refers to a group of enzymes that includes the α-amylases, the β-amylases, and the glucoamylases other than the glucoamylase from the exo-1 mutant strain of Neurospora crassa.

The term “α-amylases” (α-D-1,4-glucan glucanohydrolases) as used herein refers to a group of endohydrolases that cleave α-D-1,4-glucosidic bonds and can bypass but cannot hydrolyze α-D-1,6-glucosidic branch points. This group of enzymes shares a number of common characteristics such as a (β/α)₈ barrel structure, the hydrolysis or formation of glucosidic bonds in the α configuration, and a number of conserved amino acid residues in the active site. Several α-amylases also contain a raw-starch binding site. It is believed that Ca⁺⁺ is required for heat stability of most α-amylases. Examples of α-amylases that may be used in the present method include, but are not limited to, α-amylases from the following microorganisms, plants, and animals:
Aeromonas hydrophila
Alteromonas haloplanktis
Dictyoglomus thermophilum
Escherichia coli
Bacillus amyloliquefaciens
Bacillus megaterium
Bacillus sp. (strain B1018)
Bacillus circulans
Bacillus stearothermophilus
Bacillus licheniformis
Bacillus subtilis
Paenibacillus polymyxa (Bacillus polymyxa)
Butyrivibrio fibrisolvens
Methanococcus jannaschii
Streptomyces lividans
Streptomyces violaceus (Streptomyces venezuelae)
Streptomyces griseus
Streptomyces limosus (Streptonyces albidoflavus)
Streptoinyces hygroscopicus
Streptomyces thermoviolaceus
Colstridium acetobutylicum
Therinoanaerobacter thermosulfurogenes (Clostridium thermosulfurogenes)
Thermoanaerobacter ethanolicus (Clostridium thermohydrosulfuricum)
Thermoanaerobacter thermohydrosulfuricus (Clostridium thermohydrosulfuricum)
Thermoanaerobacter saccharolyticum
Thermononospora curvata
Pyrococcus furiosus
Pyrococcus horikoshii
Salinonalla typhimurium
Aspergillus niger
Aspergillus awamori
Aspergillus oryzae
Aspergillus shirousami
Schizosaccharomyces pombe (Fission yeast)
Saccharomycopsis fibuligera (Yeast)
Debaryomyces occidentalis (Yeast) (Schwanniomyces occidentalis)
Oryza sativa (Rice)
Triticum aestivum (Wheat)
Hordeum vulgar (Barley)
Vigna mungo (Rice bean) (Black gram)
Drosophila melanogaster (Fruit fly)
Drosophila mauritiana
Drosophila yakuba
Aedes aegypti (Yellowfever mosquito)
Dermatophagoides pteronyssinus (House-dust mite)
Tribolium castaneum (Redflour beetle)
Pecten maximus (King scallop) (Pilgrim's clam)
Tenebrio molitor (Yellow mealworm)
Porcine (Pig),
Homo sapiens (Human)
Rattus norvegicus (Rat)
Mus muscluas (Mouse)

Various manufacturers use different approaches to starch liquefaction using α-amylases but the principles are the same. Granular starch is slurried at 30-40% (w/w) with cold water, at pH 6.0-6.5, containing 20-80 ppm Ca⁺⁺ (which heat stabilizes the enzyme) and the enzyme is added (via a metering pump). The α-amylase is usually supplied at high activities so that the enzyme dose is 0.5-0.6 kg tonne⁻¹ (about 1500 U kg⁻¹ dry matter) of starch. The liquefied starch is usually saccharified but comparatively small amounts may be spray-dried for sale as ‘maltodextrins’ to the food industry mainly for use as bulking agents and in baby food. In this case, residual enzymatic activity may be destroyed by lowering the pH towards the end of the heating period.

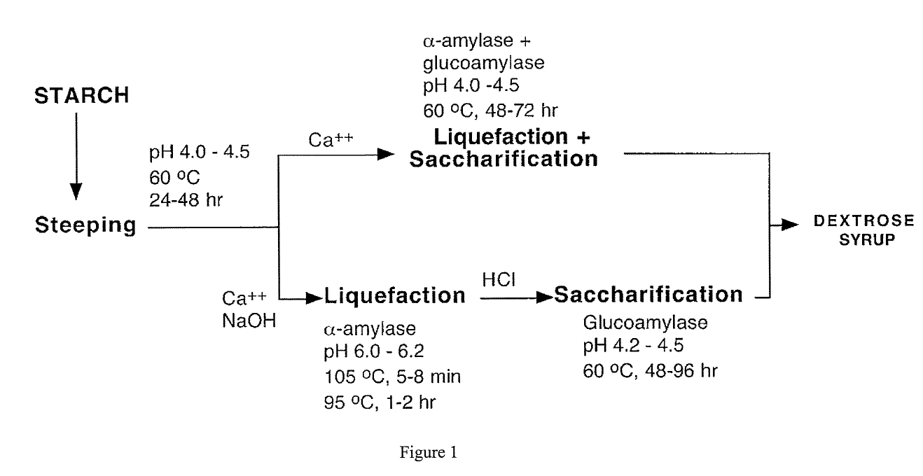

Depending on the relative location of the bond under attack as counted from the end of the chain, the products of these processes include dextrin, maltotriose, maltose, and glucose. Dextrins are shorter, broken starch segments that form as the result of the random hydrolysis of internal glucosidic bonds. A molecule of maltotriose is formed if the third bond from the end of a starch molecule is cleaved; a molecule of maltose is formed if the point of attack is the second bond; a molecule of glucose results if the bond being cleaved is the terminal one; and so on. As can be seen in FIG. 1, the initial step in random depolymerization is the splitting of large chains into various smaller sized segments. The breakdown of large molecules drastically reduces the viscosity of gelatinized starch solution. This process is called “liquefaction” because of the thinning of the solution. The final stages of depolymerization are mainly the formation of mono-, di-, and tri-saccharides. This process is called “saccharification”, due to the formation of saccharides.

The term “β-amylases” as used herein refers to enzymes that catalyze the liberation of maltose from the nonreducing ends of starch. The enzyme has a strict specificity to produce β-anomeric maltose and has been classified as a typical inverting enzyme together with glucoamylase by Koshland. β-amylases have a molecular weight of 50-60 kd and are distributed in higher plants such as soybean, sweet potato and barley, and in some microorganisms. The enzyme properties of bacterial β-amylases are different from that of the plant enzyme in optimum pH and in their ability to digest raw starch granules.

Since the three-dimensional structure of soybean β-amylase was first determined, the structures of the enzymes from sweet potato, barley and Bacillus cereus have been clarified. It was found that Bacillus cereus β-amylase contains a C-terminal starch binding domain instead of a long C-terminal loop found in the higher plant enzymes. The core structure of β-amylases from sweet potato, barley and Bacillus cereus are composed of (β/α)₈-barrel which has no similarity with that of the α-amylase family enzyme. Examples of β-amylases that may be used in the present methods inchlde, but are not limited to, β-amylases from the following microorganisms and plants:
Arabidopsis thaliana (Mouse-ear cress)
Bacillus firmus
Zea mays (Maize)
Secale cereale (Rye)
Trifolium repens (Creeping white clover)
Bacillus cereus
Hordeum vulgare (Barley)
Medicago sativa (Alfalfa)
Glycine max (Soybean)
Vigna unguiculata (Cowpea)
Bacillus circulans
Ipomoea batatas (Sweet potato)
Paenibacillus polymyxa (Bacillus polymyxa)
Thermoanaerobacter thermosulfurogenes (Clostridium thermosulfurogenes)
Triticum aestivum (wheat)

The term “glucoarnylase” (α-1,4-glucan glucohydrolase: EC 3.2.1.3) as used herein refers to an exoglucosidase that catalyzes the hydrolysis of α-1,4 bonds releasing glucose units from the non-reducing end of a starch substrate. The enzyme also acts on α-D-1,6 bonds at the branch point, although hydrolysis occurs at a slower rate. Glucoamylase is extensively used for saccharification of soluble starch in the industrial production of sweeteners and bioethanol. The hydrolysis reaction proceeds via a single-displacement mechanism involving general acid base catalysis. The end product is glucose in the β conformation. Examples of glucoamylases that may be used in the present methods include, but are not limited to, glucoamylases from the following microorganisms and plants:
Arxula adeninivorans (Yeast),
Aspergillus niger
Candida albicans (Yeast)
Hormoconis resinae (Creosote fungus)
Saccaromycopsis fibuligera (Yeast)
Saccharoinyces diastaticus (Yeast)
Maltase-glucoamylase, intestinal
Aspergillus awamori
Aspergillus oryzae
Clostridium sp. (strain G000.5)
Schizosaccharomyces pombe (Fission yeast)
Sacchormycopsis fibuligera (Yeast)
Aspergillus kawachi (Aspergillus awamor var. kawachi)
Aspergillus shrousami
Debaryomyces occidentalis (Yeast) (Schwannioinyces occidentalis)
Rhizopus oryzae (Rhizopus delemar)
Saccharoinyces cerevisiae (Baker's yeast)
Saccharomyces diastaticus (Yeast)

In certain embodiments, the glucoamylase may be the glucoamylase from Lentinula edodes (Shiitake mushrooms).

The term “isolated” as used herein refers to a molecule that has been removed from its original environment. For example, a naturally occurring protein molecule present in a living organism is not isolated, but the same protein molecule, separated from some or all of the coexisting materials in the natural system, is isolated. Such a protein can be separated from its original environment using protein purification procedures known in the art or by preparing the protein using an isolated nucleic acid that encodes the protein and recombinant procedures known in the art.

The term “starch substrate” as used herein refers to a substrate containing amylose, amylopectin, the naturally-occurring starch molecules that are found in plants such as corn, potato, rice, wheat, etc., modified starch molecules, and/or intermediates of starch hydrolysis. Examples of modified starch include, but are not limited to, starch that has been modified through partial hydrolysis, cross-linking, substitution, dextrinization, etc. Examples of intermediates of starch hydrolysis include, but are not limited to, dextrin, maltodextrin, corn syrup, etc. The term “starch substrate” as used herein also encompasses flour which contains other ingredients such as gluten, oil, fiber, etc. In certain embodiments, the starch substrate is a raw starch granule, i.e., a starch granule that has not been treated. In certain embodiments, the starch substrate is the material that is formed when starch granules, e.g. corn starch granules, are cooked, a process that releases amylose and amylopectin to some degree. In certain embodiments the starch substrate is a cereal starch such as corn, rice, wheat, barley, oats, sorghum (milo), rye or triticale starch. Each of these cereal starches inchldes several varieties. For example, corn starch includes normal (dent) starch which contains about 20-25% amylose, waxy corn starch which contains about 0% ainylose, and high amylose corn starch which contains about 50% or more amylose. In certain embodiments, the starch substrate is a root and tuber starch such as potato, tapioca (also known as Cassava), yam, sweet potato and Canna starch. In certain embodiments, the starch substrate is a legume starch such as a wrinkled pea or smooth pea starch.

### Methods of Use

Provided herein are methods for enhancing the rate of hydrolysis of a starch substrate by an amylase family enzyme or a combination of several amylase family enzymes. In one embodiment, the methods comprise contacting the starch substrate with one or more amylase family enzymes in the presence of greater than 0.001 mM Mn⁺⁺, provided that the enzyme is not the glucoamylase from the exo-1 mutant of N. crassa. In another embodiment, the method comprises the steps of a) providing a reaction mixture comprising at least one starch substrate, one or more amylase family enzymes, provided that the enzyme is not the glucoamylase from the exo-1 mutant of N. crassa, and greater than 0.001 mM Mn⁺⁺, and b) maintaining the reaction mixture under conditions that allow the one or more amylase family enzymes to catalyze the hydrolysis of at least some of the α-1,4 bonds and/or the α-1,6 bonds in the starch substrate. The hydrolysis of the bonds can be monitored by assaying for an increase in the level of reducing sugar in the reaction mixture. In certain embodiments, the reaction mixture is maintained at a temperature of from 0 to 120° C. for a time sufficient for at least 1%, 2%, 3%, 4%, 5%, 10% or more of the bonds to be hydrolyzed. In another embodiment, the methods comprise contacting a starch substrate with at least one β-amylase and/or at least one glucoamylase in the presence of greater than 0.001 mM Ca⁺⁺. In certain embodiments, the method comprises contacting a starch substrate with a β-amylase in the presence of greater than 0.001 mM manganese ion, calcium ion, magnesium ion, strontium ion, barium ion or any combination of said metal ions. In certain embodiments the method comprises contacting a starch substrate with a glucoamylase in the presence of greater than 0.001 mM manganese ion, calcium ion, lithium ion, potassium ion, or any combination of said metal ions. Without being limited by theory, the methods are based, at least in part, on discoveries by the inventors that a) the addition of greater than 0.001 mM Mn⁺⁺ to a reaction mixture comprising a starch substrate and at least one of the amylase family enzymes increases the rate of hydrolysis of the starch substrate as compared to a reaction in which Mn⁺⁺ is not included in the reaction mixture, b) the addition of greater than 0.001 mM Ca⁺⁺ to a reaction mixture comprising a starch substrate and at least one β-amylase or at least one glucoamylase increases the rate of hydrolysis of the starch substrate as compared to a reaction in which Ca⁺⁺ is not included in the reaction mixture, c) the addition of greater than 0.001 mM Mg⁺⁺, Sr⁺⁺, or Ba⁺⁺ to a reaction mixture comprising a starch substrate and at least one β-amylase enzyme increases the rate of hydrolysis of the starch substrate as compared to a reaction in which none of these cations is included in the reaction mixture, and d) the addition of greater than 0.001 mM lithium ion or potassium ion to a reaction mixture comprising a starch substrate and at least one glucoamylase enzyme increases the rate of hydrolysis of the starch substrate as compared to a reaction in which neither of these cations is included in the reaction mixture.

### Reaction Mixture

The reaction mixture used in the present methods preferably has a pH of 8 or less. In certain embodiments, the pH of the reaction mixture is from 4 to 7. In certain embodiments, the pH of the reaction mixture is from 4 to 6. In certain embodiments, the reaction mixture has a pH of 5 to 5.5.

In certain embodiments, the reaction mixture comprises a starch substrate, at least one amylase family enzyme, more than 0.001 mM Mn⁺⁺, and preferably a buffer. In certain embodiments, the reaction mixture comprises from 0.01 mM to 100 mM Mn⁺⁺. In certain embodiments, the reaction mixture comprises from 0.01 mM to 50 mM Mn⁺⁺. In certain embodiments, the reaction mixture comprises from 0.01 mM to 20 mM Mn⁺⁺. In certain embodiments, the reaction mixture comprises from 0.01 mM to 10 mM Mn⁺⁺. In certain embodiments, the reaction mixture comprises from 0.1 mM to 10 mM Mn⁺⁺. In certain embodiments, the reaction mixture comprises from 0.1 mM to 1 mM Mn⁺⁺. In certain embodiments, the reaction mixture comprises from 1.0 mM to 10 mM Mn⁺⁺. The manganese ions can be provided in the form of any manganese salt including, but not limited to, manganese chloride, manganese acetate, manganese sulfate, manganese bromide, manganese difluoride, manganese nitrate, manganese oxalate, manganese benzoate, manganese phosphate and manganese phosphate dibasic. In certain embodiments, the manganese ion is pre-incubated with the enzyme for 1, 2, 3, 4, 5, 6, 7, 8, 9, 10, or more minutes at 0-120° C., 10-110° C. or 20-100° C. prior to combining with the starch substrate.

In certain embodiments, the reaction mixture comprises a starch substrate, at least one glucoamylase and/or at least one β-amylase, more than 0.001 mM Ca⁺⁺, and preferably a buffer. In certain embodiments, the reaction mixture comprises from 0.01 mM to 100 mM Ca⁺⁺. In certain embodiments, the reaction mixture comprises from 0.1 mM to 100 mM Ca⁺⁺. In certain embodiments, the reaction mixture comprises from 0.1 mM to 50 mM Ca⁺⁺. In certain embodiments, the reaction mixture comprises from 0.1 mM to 20 mM Ca⁺⁺. In certain embodiments, the reaction mixture comprises from 0.1 mM to 10 mM Ca⁺⁺. The calcium ions can be provided in the form of any calcium salt including, but not limited to, calcium acetate, calcium acetylsalicylate, calcium ascorbate, calcium bromide, calcium borogluconate, calcium chloride, calcium formate, calcium iodide, calcium nitrate, calcium succinate, and calcium sulfate. In certain embodiments, the reaction mixture comprises both manganese and calcium ions.

The amount of starch substrate in the reaction mixture depends on the type(s) of enzymes included in the reaction mixture, the purity of the enzymes, the degree of hydrolysis desired, reaction conditions such as temperature, pH and time. In certain embodiments, the starch substrate is derived from corn. Suitable corn starch substrates are raw corn starch, soluble corn starch, dent corn starch, high amylose corn starch and waxy corn starch. In another embodiment, the starch substrate is derived from rice. In another embodiment, the starch substrate is derived from potato. In another embodiment, the starch substrate is derived from barley. In another embodiment, the starch substrate is derived from wheat. In another embodiment, the starch substrate is a tapioca starch. In another embodiment the starch substrate is a sorghum (milo) starch. In certain embodiments, the starch substrate is a cooked or gelatinized starch. In another embodiment, the starch substrate is an intermediate of starch hydrolysis. In another embodiment, the starch substrate is a modified starch substrate. Examples of modified starches include, but are not limited to, pre-gelatinized starch (heat treatment), thin boiled starch (acid treatment), acetylated starch, oxidized starch, hydroxy propylated starch, hydroxyl ethylated starch, octenyl succinate starch, carboxy methyl starch, and cationic starch.

The amount of enzyme in the reaction mixture depends on factors such as the type(s) of enzymes included in the reaction mixture, the purity of the enzyme(s), the amount and type of starch in the reaction mixture, and reaction conditions such as temperature, pH and reaction time, and can be determined by one skilled in the art using routine experimentation.

### Conditions

The reaction mixture is maintained at an appropriate temperature for a time sufficient for hydrolysis of at least a portion (e.g. 1%, 2%, 3% or more) of the starch substrate to occur. In certain embodiments, the reaction mixture is incubated at a temperature greater than 0 and less than 120° C. In certain embodiments, the reaction mixture is incubated at a temperature of from 20° C. to 110° C. In certain embodiments such as when an α-amylase is included in the reaction mixture, the mixture may be incubated at a temperature of from about 50 to 110° C. In certain embodiments, such as when glucoamylase is included in the reaction mixture, the mixture may be incubated at a temperature of about 50-65° C. In certain embodiments such as when a β-amylase is included in the reaction mixture, the mixture may be incubated at a temperature of from about 55 to 60° C. In certain embodiments, the reaction mixture is maintained at the appropriate temperature for at least 5 minutes. In other embodiments, the reaction mixture is maintained at the appropriate temperature until hydrolysis is substantially complete. Methods for determining % of hydrolysis include assays which determine the amount of reducing sugar in the reaction mixture, such as the spectrophotometric method described in the examples below and high performance liquid chromatography (HPLC).

The invention may be better understood by reference to the following examples, which serve to illustrate but not to limit the present invention.

### EXAMPLES

### Example 1

An enzyme mixture of purified glucoamylase from mushroom and purified α-amylase from barley was pre-incubated in sodium acetate buffer, pH 5.3, comprising 0.01 mM, 0.1 mM, 1.0 mM, or 10 mM Mn⁺⁺ for 20 min at 20° C. Enzyme mixture was also pre-incubated with 10 mM Fe⁺⁺, Cu⁺⁺, Ca⁺⁺, Mg⁺⁺, or disodium ethylenediamine-tetraacetic acid (EDTA). Thereafter, 40 μl of each pre-incubation solution was added to 15 mg raw corn starch, and the reaction mixture incubated at 37° C. for 20 minutes. The control reaction mixture contained substrate, enzyme, and buffer but no additional metal ions. The starch hydrolysis reaction was stopped by combining the reaction mixture with a solution containing 3,5 dinitrosalicylic acid (DNSA) and heating for 30 min at 85° C. The absorbance of the control and test samples was measured at 562 nm to monitor the amount of reducing sugar in each sample. The results, in terms of relative activity of the test samples, which is determined by comparing the absorbance reading of the test sample to the absorbance reading of the corresponding control sample, whose corrected absorbance reading is set at a value of 100% relative activity, are shown in Table 2 below.

TABLE 2

| | | Other Chemical | |
|---|---|---|---|
| | Relative | Additives | Relative |
| Mn⁺⁺ (mM) | Activity (%) | 10 mM | Activity (%) |
| 0 (Control) | 100 | Fe⁺⁺ | 74.0 |
| 0.01 | 106.7 | Cu⁺⁺ | 47.5 |
| 0.1 | 124.7 | Ca⁺⁺ | 97.3 |
| 1 | 142.6 | Mg⁺⁺ | 92.4 |
| 10 | 143.5 | Disodium EDTA | 88.8 |

These results show that Mn⁺⁺ enhances the hydrolysis of raw corn starch by an enzyme mixture of α-amylase (purified from barley) and glucoamylase (purified from mushroom), while the other additives inchlding Fe⁺⁺, Cu⁺⁺, Ca⁺⁺, Mg⁺⁺, and EDTA do not have a similar effect.

### Example 2

The α-amylase from Aspergillus oryzae and the glucoamylase from Rhizopus sp. Each were separately pre-incubated in a 25 mM sodium acetate buffer, pH 5.3, comprising 0.01 mM, 0.1 mM, 1.0 mM, or 10 mM Mn⁺⁺ for 20 min at 20° C. Thereafter, 40 μl of each pre-incubation solution was added to 15 mg raw corn starch, and the reaction mixture incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 3 below.

TABLE 3

| | α-Amylase | Glucoamylase |
|---|---|---|
| Mn⁺⁺ | Relative Activity | Relative Activity |
| (mM) | (%) | (%) |
| 0 (Control) | 100 | 100 |
| 0.01 | 106.6 | 105.0 |
| 0.1 | 125.5 | 119.2 |
| 1 | 136.7 | 133.5 |
| 10 | 155.6 | 162.6 |

These results show that Mn⁺⁺ enhances the hydrolysis of raw corn starch by Aspergillus oryzae α-amylase and Rhizopus sp. Glucoamylase.

### Example 3

The α-amylase from Bacillus amyloliquefaciens the α-amylase from Bacillus licheniformis and the glucoamylase from Aspergillus Niger were each separately pre-incubated in a 25 mM sodium acetate buffer, pH 5.3, comprising 0.01 mM, 0.1 mM, 1.0 mM, or 10 mM Mn⁺⁺ for 20 min at 20° C. Thereafter, 40 μl of each pre-incubation solution was added to 15 mg raw corn starch, and the reaction mixture incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 4 below.

TABLE 4

| | Bacillus | Bacillus | Aspergillus |
|---|---|---|---|
| | amyloliquefaciens | licheniformis | niger |
| | α-amylase | α-amylase | glucoamylase |
| | Relative | Relative | Relative |
| Mn⁺⁺ (mM) | Activity (%) | Activity (%) | Activity (%) |
| 0 (Control) | 100 | 100 | 100 |
| 0.01 | 101.5 | 93.4 | 94.4 |
| 0.1 | 115.7 | 104.6 | 111.2 |
| 1 | 119.1 | 113.8 | 127.1 |
| 10 | 111.7 | 122.3 | 160.6 |

These results show that Mn⁺⁺ enhances the hydrolysis of raw corn starch by Bacillus amyloliquefaciens α-amylase, Bacillus licheniformis α-amylase, and Aspergillus niger glucoamylase.

### Example 4

The α-amylase from bovine pancreas was pre-incubated in a 25 mM sodium acetate buffer at pH 5.3 or in a potassium phosphate buffer, at pH 7.0 with 0.01 mM, 0.1 mM, 1.0 mM, or 10 mM Mn⁺⁺ for 20 min at 20° C. Thereafter, 40 μl of each pre-incubation solution was added to 15 mg raw corn starch, and the reaction mixture incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 5 below.

TABLE 5

| Mn⁺⁺ | Relative Activity (%) | Relative Activity (%) |
|---|---|---|
| (mM) | pH 7.0 | pH 5.3 |
| 0 (control) | 100 | 100 |
| 0.01 | 103.5 | 108.3 |
| 0.1 | 103.5 | 125.0 |
| 1 | 119.6 | 145.2 |
| 10 | 95.7 | 139.9 |

These results show that Mn⁺⁺ activation on the hydrolysis of raw corn starch by pancreatic α-amylase depends on the pH of the reaction, with greater activation at a pH of 5.3 than at 7.0.

### Example 5

Solutions of 25 mM sodium acetate buffer, pH 5.3, containing 0.01 mM, 0.1 mM, 1.0 mM, or 10 mM Mn⁺⁺ were prepared. Thereafter 40 μl of the buffer and metal solution was added to 15 mg raw corn starch and the resulting mixture was pre-incubated at 20° C. for 20 minutes. The starch mixture was washed 5 times with excess buffer, followed by centrifugation, and removal of the supernatant. Then an enzyme solution containing buffer and the glucoamylase from Aspergillus niger was added to the washed starch and the reaction mixture incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 6 below.

TABLE 6

| Mn⁺⁺ (mM) | Relative Activity (%) |
|---|---|
| 0 (Control) | 100 |
| 0.01 | 89.1 |
| 0.1 | 84.2 |
| 1 | 95.0 |
| 10 | 89.1 |

These results indicate that Mn⁺⁺-induced enhancement on the enzymatic hydrolysis of raw corn starch is not caused by the action of Mn⁺⁺ on the starch.

### Example 6

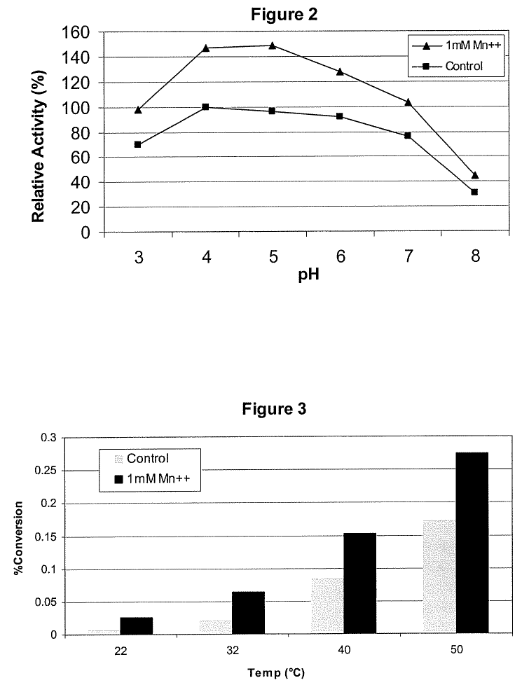

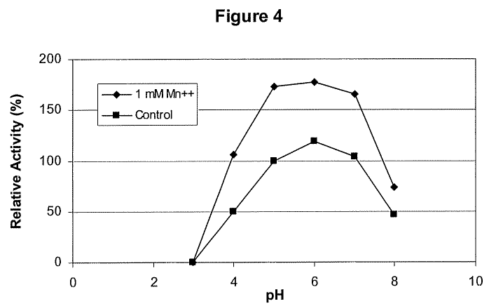

Enzyme solutions of the glucoamylase from Rhizopus sp. Were prepared in various pH buffers having a pH of 3, 4, 5, 6, 7, or 8. Thereafter, 40 μl of the buffered enzyme solution and a stock solution containing Mn⁺⁺ were added to 6 samples containing 15 mg raw corn starch to provide a reaction mixture containing 1.0 mM Mn⁺⁺. The reaction mixtures were incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in FIG. 2 and Table 7 below.

TABLE 7

| | Control | Mn⁺⁺ |
|---|---|---|
| pH | Relative Activity (%) | Relative Activity (%) |
| 3 | 70.2 | 98 |
| 4 | 100 | 146.7 |
| 5 | 96.5 | 148.6 |
| 6 | 91.8 | 127.8 |
| 7 | 75.7 | 103.1 |
| 8 | 30.6 | 44.3 |

These results show that the Mn⁺⁺-induced activation on the hydrolysis of raw corn starch by Rhizopus sp. Glucoamylase is the greatest at a pH of 5.0.

### Example 7

An enzyme solution of the glucoamylase from Rhizopus sp. Was prepared in a sodium acetate buffer, pH 5.3 comprising 1.0 mM Mn⁺⁺. Thereafter, 50 μl of the enzyme solution was added to each of four samples containing 15 mg raw corn starch and incubated at 22° C., 32° C., 40° C. or 50° C., respectively, for 10 minutes. The control reaction mixture contained substrate, enzyme, and buffer but no manganese ion. The starch hydrolysis reaction was stopped by combining each reaction mixture with a solution containing DNSA and heating for 30 min at 85° C. The absorbance of the control and test samples was measured at 562 nm to monitor the amount of reducing sugar in each sample. The results, in terms of percent conversion of the starch substrate at various temperatures, are shown in FIG. 3 and Table 8 below.

TABLE 8

| | Control (no Mn⁺⁺) | 1 mM Mn⁺⁺ |
|---|---|---|
| Temp (° C.) | % Conversion | % Conversion |
| 22 | 0.007 | 0.026 |
| 32 | 0.021 | 0.064 |
| 40 | 0.084 | 0.152 |
| 50 | 0.171 | 0.274 |

These results show that Mn⁺⁺ activation on the hydrolysis of raw corn starch by Rhizopus sp. Glucoamylase occurs at a wide temperature range.

### Example 8

An enzyme solution of the glucoamylase from Rhizopus sp. Was prepared in a sodium acetate buffer, pH 5.3, comprising 1.0 mM manganese chloride, manganese acetate, or manganese sulfate. Thereafter, 40 μl of each enzyme solution was added to 15 mg raw corn starch and the reaction mixtures incubated at 37° C. for 5, 10, 15, 20, or 25 minutes. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of % conversion of the starch substrate, are shown in Table 9 below.

TABLE 9

| | Control | MnCl₂ | MnOAc | MnSO₄ |
|---|---|---|---|---|
| Time | % conversion | % Conversion | % Conversion | % Conversion |
| 0 | 0 | 0 | 0 | 0 |
| 5 | 0.32 | 0.44 | 0.46 | 0.50 |
| 10 | 0.49 | 0.74 | 0.74 | 0.87 |
| 15 | 0.70 | 1.01 | 1.04 | 1.12 |
| 20 | 0.89 | 1.22 | 1.28 | 1.30 |
| 25 | 1.20 | 1.70 | 1.50 | 1.58 |

These results show that the anion species of manganese do not affect the activation on the hydrolysis of raw corn starch by Rhizopus sp. Glucoamylase.

### Example 9

The glucoamylase from Rhizopus sp. Or raw corn starch substrate were pre-incubated in a sodium acetate buffer, pH 5.3, with 1.0 mM Mn⁺⁺ for 0, 20, 40, or 60 min at 22° C. The enzyme pre-incubation solutions were then combined with 15 mg raw corn starch to provide a reaction mixture that was incubated at 37° C. for 5 minutes. The starch pre-incubation solutions were combined with an enzyme solution containing the glucoamylase from Rhizopus sp and buffer to provide a reaction mixture that was incubated at 37° C. for 5 minutes or 10 minutes. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The absorbance was measured to monitor the amount of reducing sugar in each sample. The results in terms of % conversion of the starch substrate are shown in Table 10 below.

TABLE 10

| Time | Mn⁺⁺ + GLA* | Mn⁺⁺ + RCS** | Mn⁺⁺ + RCS** |
|---|---|---|---|
| (min) | 37° C., 5 min | 37° C., 10 min | 37° C., 5 min |
| 0 | 0.42 | 0.73 | 0.51 |
| 20 | 0.35 | 0.79 | 0.48 |
| 40 | 0.38 | 0.81 | 0.50 |
| 60 | 0.38 | 0.80 | 0.58 |

*GLA—Glucoamylase
**RCS—Raw Corn Starch

These results show that pre-incubation of Mn⁺⁺ with enzyme or pre-incubation of Mn⁺⁺ with starch does not significantly influence the activation effect of Mn⁺⁺ on the enzymatic hydrolysis of raw corn starch.

### Example 10

Enzyme solutions of the β-amylase from sweet potato (Sigma A-7005) and the β-amylase Type II-B from barley (Sigma-7130) were prepared in a 25 mM sodium acetate buffer, pH 5.3, comprising 0.01 mM, 0.1 mM, 1.0 mM, or 10 mM Mn⁺⁺. Each of the buffered enzyme solutions was separately added to 15 mg raw corn starch or 0.6 mg soluble starch, and the reaction mixtures incubated at 37° C. for 20 minutes. The control reaction mixture contained substrate, enzyme, and buffer but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of samples, are shown in Table 11 below.

TABLE 11

| | | | | β-amylase |
|---|---|---|---|---|
| | β-amylase | β-amylase | β-amylase | from |
| | from barley + | from barley + | from potato + | potato + |
| | *RCS | **SS | *RCS | **SS |
| Mn⁺⁺ | % Relative | % Relative | % Relative | % Relative |
| (mM) | Activity | Activity | Activity | Activity |
| 0 | 100 | 100 | 100 | 100 |
| 0.01 | 99.5 | 112.5 | 113.9 | 108.6 |
| 0.1 | 118.4 | 119.2 | 106.6 | 127.1 |
| 1 | 133.3 | 155.7 | 136.5 | 146.6 |
| 10 | 152.2 | 157.3 | 160.6 | 176.3 |

*RCS—Raw corn starch
**SS—Soluble starch

These results show that Mn⁺⁺ enhances the hydrolysis of both raw corn starch and soluble starch by barley and sweet potato β-amylases.

### Example 11

The α-amylase from Bacillus licheniformis was added to a sodium acetate buffer solution, pH 5.2, comprising 0.01 mM, 0.1 mM, 1.0 mM, or 10 mM Mn⁺⁺ and 0.5% by weight cooked potato starch, soluble potato starch, wheat starch, rice starch, barley starch, pea starch, or tapioca starch, and the separate reaction mixtures were then incubated at 37° C. for 10 minutes. The control reaction mixture contained enzyme, buffer, and starch but no manganese ion. The starch hydrolysis reaction was stopped by combining the reaction mixture with 3,5 dinitrosalicylic acid (DNSA) and heating for 30 min at 85° C. The absorbance of the control and test samples was measured at 562 nm to monitor the amount of reducing sugar in each sample. The results, in terms of relative activity of the samples, are shown in Tables 12 and 13 below.

TABLE 12

Relative Activity (%)

| | Soluble Potato | Wheat | Rice | Tapioca |
|---|---|---|---|---|
| Mn⁺⁺ (mM) | Starch | Starch | Starch | Starch |
| 0 (control) | 100.0 | 100.0 | 100.0 | 100.0 |
| 0.01 | 118.3 | 118.7 | 115.5 | 108.3 |
| 0.1 | 147.6 | 118.2 | 126.0 | 119.2 |
| 1 | 142.0 | 153.0 | 129.6 | 121.5 |
| 10 | 173.1 | 142.1 | 157.7 | 157.3 |

TABLE 13

| | Relative Activity (%) | | |
|---|---|---|---|
| Mn⁺⁺ (mM) | Potato Starch | Barley Starch | Pea Starch |
| 0(control) | 100.0 | 100.0 | 100.0 |
| 0.01 | 109.9 | 135.3 | 138.7 |
| 0.1 | 117.5 | 129.2 | 129.5 |
| 1 | 128.7 | 117.4 | 143.9 |
| 10 | 160.2 | 142.2 | 182.3 |

### Example 12

The β-amylase from barley was added to a sodium acetate buffer solution, pH 5.2, comprising 0.01 mM, 0.1 mM, 1.0 mM, or 10 mM Mn⁺⁺ and 0.5% by weight cooked potato starch, soluble potato starch, wheat starch, rice starch, barley starch, pea starch, or tapioca starch, and the separate reaction mixtures were then incubated at 37° C. for 10 minutes. The control reaction mixture contained enzyme, buffer, and starch but no manganese ion. The starch hydrolysis reaction was stopped by combining the reaction mixture with 3,5 dinitrosalicylic acid (DNSA) and heating for 30 min at 85° C. The absorbance of the control and test samples was measured at 562 nm to monitor the amount of reducing sugar in each sample. The results, in terms of relative activity of the samples, are shown in Tables 14 and 15 below.

TABLE 14

Relative Activity (%)

| | Soluble Potato | Wheat | Rice | Tapioca |
|---|---|---|---|---|
| Mn⁺⁺ (mM) | Starch | Starch | Starch | Starch |
| 0 (control) | 100.0 | 100.0 | 100.0 | 100.0 |
| 0.01 | 117.4 | 120.2 | 107.8 | 111.3 |
| 0.1 | 136.0 | 141.9 | 123.9 | 122.1 |
| 1 | 154.1 | 151.9 | 138.1 | 135.6 |
| 10 | 165.6 | 221.1 | 173.4 | 180.5 |

TABLE 15

| | Relative Activity (%) | | |
|---|---|---|---|
| Mn⁺⁺ (mM) | Potato Starch | Barley Starch | Pea Starch |
| 0 (control) | 100.0 | 100.0 | 100.0 |
| 0.01 | 109.5 | 115.2 | 108.1 |
| 0.1 | 117.5 | 121.1 | 122.3 |
| 1 | 135.4 | 154.0 | 139.2 |
| 10 | 175.1 | 197.5 | 253.4 |

### Example 13

The glucoamylase from Aspergillus niger was added to a sodium acetate buffer solution, pH 5.2, comprising 0.01 mM, 0.1 mM, 1.0 mM, or 10 mM Mn⁺⁺ and 0.5% by weight cooked potato starch, soluble potato starch, wheat starch, rice starch, barley starch, pea starch, or tapioca starch, and the separate reaction mixtures were then incubated at 37° C. for 10 minutes. The control reaction mixture contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in tenns of relative activity of the samples, are shown in Tables 16 and 17 below.

TABLE 16

Relative Activity (%)

| | Soluble Potato | Wheat | Rice | Tapioca |
|---|---|---|---|---|
| Mn⁺⁺ (mM) | Starch | Starch | Starch | Starch |
| 0 (control) | 100.0 | 100.0 | 100.0 | 100.0 |
| 0.01 | 115.7 | 111.9 | 109.3 | 109.5 |
| 0.1 | 130.1 | 122.2 | 129.3 | 126.0 |
| 1 | 79.9 | 142.5 | 144.0 | 135.7 |
| 10 | 118.8 | 185.4 | 209.2 | 183.8 |
| 0 (control) | 100.0 | | | |
| 0.01 | 104.0 | | | |
| 0.1 | 121.0 | | | |
| 1 | 140.0 | | | |
| 10 | 245.2 | | | |

TABLE 17

| | Relative Activity (%) | | |
|---|---|---|---|
| Mn⁺⁺ (mM) | Potato Starch | Barley Starch | Pea Starch |
| 0 (control) | 100.0 | 100.0 | 100.0 |
| 0.01 | 112.0 | 111.1 | 121.1 |
| 0.1 | 118.8 | 117.5 | 124.0 |
| 1 | 138.5 | 120.4 | 138.0 |
| 10 | 200.0 | 172.6 | 179.8 |

The results from Examples 11-13 indicate that Mn⁺⁺ increases the rate of enzymatic hydrolysis of all starches from different botanical sources by a member of each group in the amylase family of enzymes.

### Example 14

The α-amylase from Bacillus licheniformis was added to a sodium acetate buffer solution, pH 5.2, comprising 0.01 mM, 0.1 mM, 1.0 mM, or 10 mM Mn⁺⁺ and 0.5% by weight cooked soluble corn starch, dent corn starch, waxy corn starch, or high amylose V corn starch. The separate reaction mixtures were incubated at 37° C. for 10 minutes. The control reaction mixture contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 18 below.

TABLE 18

| | Relative Activity (%) | | | |
|---|---|---|---|---|
| | Soluble Corn | Dent Corn | Waxy Corn | High-amylose V |
| Mn⁺⁺ (mM) | Starch | Starch | Starch | Corn Starch |
| 0 (control) | 100.0 | 100.0 | 100.0 | 100.0 |
| 0.01 | 118.3 | 103.5 | 118.3 | 116.5 |
| 0.1 | 128.1 | 124.4 | 126.9 | 135.5 |
| 1 | 135.8 | 128.7 | 129.9 | 133.5 |
| 10 | 162.7 | 147.8 | 155.8 | 160.1 |

### Example 15

The β-amylase from barley was added to a sodium acetate buffer solution, pH 5.2, comprising 0.01 mM, 0.1 mM, 1.0 mM, or 10 mM Mn⁺⁺ and 0.5% by weight cooked soluble corn starch, dent corn starch, waxy corn starch, or high amylose V corn starch. The separate reaction mixtures were incubated at 37° C. for 10 minutes. The control reaction mixture contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 19 below.

TABLE 19

| | Relative Activity (%) | | | |
|---|---|---|---|---|
| | Soluble Corn | Dent Corn | Waxy Corn | High-amylose V |
| Mn⁺⁺ (mM) | Starch | Starch | Starch | Corn Starch |
| 0 (control) | 100.0 | 100.0 | 100.0 | 100.0 |
| 0.01 | 138.7 | 123.5 | 114.2 | 111.2 |
| 0.1 | 135.7 | 135.3 | 129.9 | 123.3 |
| 1 | 174.8 | 149.8 | 148.3 | 136.3 |
| 10 | 199.6 | 210.1 | 237.4 | 184.5 |

### Example 16

The glucoamylase from Aspergillus niger was added to a sodium acetate buffer solution, pH 5.2, comprising 0.01 mM, 0.1 mM, 1.0 mM, or 10 mM Mn⁺⁺ and 0.5% by weight cooked soluble corn starch, dent corn starch, waxy corn starch, or high amylose V corn starch. The separate reaction mixtures were incubated at 37° C. for 10 minutes. The control reaction mixture contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 20 below.

TABLE 20

| | Relative Activity (%) | | | |
|---|---|---|---|---|
| | Soluble Corn | Dent Corn | Waxy Corn | High-amylose V |
| Mn⁺⁺ (mM) | Starch | Starch | Starch | Corn Starch |
| 0 (control) | 100.0 | 100.0 | 100.0 | 100.0 |
| 0.01 | 119.1 | 113.3 | 113.0 | 119.2 |
| 0.1 | 129.1 | 126.1 | 130.1 | 130.9 |
| 1 | 144.8 | 148.4 | 147.2 | 141.3 |
| 10 | 178.3 | 186.9 | 200.3 | 193.1 |

The results from Examples 14-16 indicate that Mn⁺⁺ increases the enzymatic rate of hydrolysis of different varieties of corn starch by a member of each group in the amylase family of enzymes.

### Example 17

The α-amylase from Bacillus licheniformis, the α-amylase from Bacillus amyloliquefaciens, the α-amylase from Aspergillus oryzae, the α-amylase from porcine pancreas, the α-amylase from Bacillus subtilis, the α-amylase from human saliva, the industrial α-amylase known as Termamyl, the β-amylase from barley, the β-amylase from sweet potato, the glucoamylase from Aspergillus niger and the glucoamylase from Rhizopus sp. were each separtely added to a sodium acetate buffer solution, pH 5.2, comprising 0.01 mM, 0.1 mM, 1.0 mM, or 10 mM Mn⁺⁺ and 0.5% by weight cooked dent corn starch. The separate reaction mixtures were incubated at 37° C. for 10 minutes. The control reaction mixtures contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Tables 21-24 below.

TABLE 21

| | Relative Activity (%) | | | |
|---|---|---|---|---|
| | α-amylase, | α-amylase, | α-amylase, | α-amylase, |
| Mn⁺⁺ | Bacillus | Bacillus | Aspergillus | Porcine |
| (mM) | licheniformis | amyloliquefaciens | oryzae | Pancreas |
| 0 (control) | 100.0 | 100.0 | 100.0 | 100.0 |
| 0.01 | 103.5 | 89.0 | 126.9 | 114.5 |
| 0.1 | 124.4 | 120.4 | 131.2 | 132.4 |
| 1 | 128.7 | 97.3 | 135.2 | 144.2 |
| 10 | 147.8 | 136.5 | 145.1 | 177.7 |
| 0 (control) | | 100.0 | | |
| 0.01 | | 97.3 | | |
| 0.1 | | 121.0 | | |
| 1 | | 114.8 | | |
| 10 | | 126.5 | | |

TABLE 22

| | Relative Activity (%) | | |
|---|---|---|---|
| | α-amylase, | α-amylase, | α-amylase, |
| Mn⁺⁺ (mM) | Bacillus subtilis | Human saliva | Termamyl |
| 0 (control) | 100.0 | 100.0 | 100.0 |
| 0.01 | 119.5 | 120.2 | 114.7 |
| 0.1 | 128.2 | 147.5 | 123.6 |
| 1 | 118.4 | 224.4 | 149.8 |
| 10 | 123.9 | 275.6 | 156.3 |
| 0 (control) | 100.0 | | |
| 0.01 | 113.0 | | |
| 0.1 | 118.7 | | |
| 1 | 115.3 | | |
| 10 | 123.6 | | |

TABLE 23

Relative Activity (%)

| | β-Amylase from | β-Amylase from |
|---|---|---|
| Mn⁺⁺ (mM) | barley | Sweet potato |
| 0 (control) | 100.0 | 100.0 |
| 0.01 | 123.5 | 117.6 |
| 0.1 | 135.3 | 131.4 |
| 1 | 149.8 | 148.0 |
| 10 | 210.1 | 215.0 |

TABLE 24

Relative Activity (%)

| | Glucoamylase, | Glucoamylase, |
|---|---|---|
| Mn⁺⁺ (mM) | Aspergillus niger | Rhizopus |
| 0 (control) | 100.0 | 100.0 |
| 0.01 | 113.3 | 112.0 |
| 0.1 | 126.1 | 123.8 |
| 1 | 148.4 | 145.1 |
| 10 | 186.9 | 223.5 |

The results indicate that Mn⁺⁺ increases the enzymatic rate of hydrolysis of a starch substrate by multiple members from each group in the amylase family of enzymes.

### Example 18

The α-amylase from Bacillus licheniformis was added to a sodium acetate buffer solution, pH 5.2, comprising no metal or 10 nM Fe⁺⁺, Mg⁺⁺, Li⁺, Zn⁺⁺, Cu⁺⁺, Ca⁺⁺, di-sodium EDTA, NH₄⁺, Sr⁺⁺, Na⁺, K⁺, Al⁺⁺⁺, or Ba⁺⁺ and 0.5% by weight cooked dent corn starch. The separate reaction mixtures were incubated at 37° C. for 10 minutes. The control reaction mixture contained enzyme, buffer, and starch but no additive. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 25 below.

TABLE 25

| Additive (10 mM) | Relative Activity (%) |
|---|---|
| Fe++ | 101.9 |
| Mg++ | 83.8 |
| Li+ | 89.2 |
| Zn++ | 100.2 |
| Cu++ | 30.0 |
| Di-sodium EDTA | 103.8 |
| NH₄⁺ | 86.4 |
| Sr++ | 87.2 |
| Na+ | 84.2 |
| K+ | 82.8 |
| Al+++ | 95.1 |
| Ba++ | 98.1 |
| Ca++ | 119.1 |
| Ca++ | 94.8 |
| Ca++ | 99.4 |
| Ca++ | 90.0 |
| Ca++ (1 mM) | 83.6 |

The results indicate that 10 mM Fe⁺⁺, Mg⁺⁺, Li⁺, Zn⁺⁺, Cu⁺⁺, di-sodium EDTA, NH₄⁺, Sr⁺⁺, Na⁺, K⁺, Al⁺⁺⁺, or Ba⁺⁺ does not increase the enzymatic rate of hydrolysis of a starch substrate by a member of α-amylase family.

### Example 19

The β-amylase from barley was each added to a sodium acetate buffer solution, pH 5.2, comprising no metal or 10 mM Fe⁺⁺, Mg⁺⁺, Li⁺, Zn⁺⁺, Cu⁺⁺, Ca⁺⁺, di-sodium EDTA, NH₄⁺, Sr⁺⁺, Na⁺, K⁺, Al⁺⁺⁺, or Ba⁺⁺ and 0.5% by weight cooked dent corn starch. The separate reaction mixtures were incubated at 37° C. for 10 minutes. The control reaction mixture contained enzyme, buffer, and starch but no additive. The starch hydrolysis reaction was stopped by combining the reaction mixture with a solution containing DNSA and heating for 30 min at 85° C. The absorbance of the control and test samples was measured at 562 nM to monitor the amount of reducing sugar in each sample. The results, in terms of relative activity of the samples, are shown in Table 26 below.

TABLE 26

| Additive (10 mM) | Relative Activity (%) |
|---|---|
| Fe++ | 88.7 |
| Mg++ | 110.8 |
| Li+ | 101.6 |
| Zn++ | 70.6 |
| Cu++ | 6.7 |
| EDTA | 100.4 |
| NH₄⁺ | 95.1 |
| Sr++ | 114.1 |
| Na+ | 95.1 |
| K+ | 101.1 |
| Al+++ | 77.5 |
| Ba++ | 114.6 |
| Ca++ | 114.1 |
| Ca++ | 127.2 |
| Ca++ | 121.2 |
| Ca++ (1 mM) | 111.8 |
| Ca++ (1 mM) | 105.8 |
| Ca++ (1 mM) | 105.0 |

The results indicate that 10 mM Mg⁺⁺, Sr⁺⁺, Ca⁺⁺, and Ba⁺⁺ increase the rate of enzymatic hydrolysis of a starch substrate with a β-amylase from barley, while 10 mM Fe⁺⁺, Li⁺, Al⁺⁺⁺, Zn⁺⁺, Cu⁺⁺, EDTA, NH₄⁺, Na⁺, and K⁺ do not significantly increase the rate of enzymatic hydrolysis of a starch substrate by the β-amylase from barley.

### Example 20

The glucoamylase from Aspergillus Niger was added to a sodium acetate buffer solution, pH 5.2, comprising no metal or 10 mM Fe⁺⁺, Mg⁺⁺, Li⁺, Zn⁺⁺, Cu⁺⁺, Ca⁺⁺, di-sodium EDTA, NH₄⁺, Sr⁺⁺, Na⁺, K⁺, Al⁺⁺⁺, or Ba⁺⁺ and 0.5% by weight cooked dent corn starch. The separate reaction mixtures were incubated at 37° C. for 10 minutes. The control reaction mixture contained enzyme, buffer, and starch but no additive. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples, are shown in Table 27 below.

TABLE 27

| Additive (10 mM) | Relative Activity (%) |
|---|---|
| Fe++ | 98.3 |
| Mg++ | 96.0 |
| Li+ | 118.1 |
| Zn++ | 57.3 |
| Cu++ | 33.9 |
| EDTA | 57.6 |
| NH₄⁺ | 98.3 |
| Sr++ | 93.8 |
| Na+ | 97.3 |
| K+ | 113.3 |
| Al+++ | 96.2 |
| Ba++ | 99.3 |
| Ca++ | 103.8 |
| Ca++ | 104.4 |
| Ca++ | 97.4 |
| Ca++ (1 mM) | 108.0 |

The results indicate that 10 mM Li⁺, 10 mM K⁺, and 10 mM Ca⁺⁺ increase the rate of enzymatic hydrolysis of a starch substrate by a glucoamylase enzyme, as compared to a control which does not include these cations. The results also indicate that 10 mM Fe⁺⁺, Mg⁺⁺, Zn⁺⁺, Cu⁺⁺, di-sodium EDTA, NH₄⁺, Sr⁺⁺, Na⁺, Al⁺⁺⁺, or Ba⁺⁺ do not significantly increase the rate of enzymatic hydrolysis of a starch substrate by the glucoamylase from Aspergillus niger.

### Example 21

β-amylase from barley enzyme solution was added to a mixture comprising 1 mM Mn⁺⁺, sodium acetate buffer solution and 0.5% by weight cooked dent corn starch at pH 3, 4, 5, 6, 7, or 8. The separate reaction mixtures were then incubated at 37° C. for 10 minutes. The control reaction mixture contained enzyme, buffer, and starch but no manganese ion. The starch hydrolysis reaction was stopped by combining the reaction mixture with a solution containing DNSA and heating for 30 min at 85° C. The absorbance of the control and test samples was measured at 562 nm to monitor the amount of reducing sugar in each sample. The results, in terms of relative activity of the samples are shown in Table 28.

TABLE 28

| | Control | |
|---|---|---|
| | Relative Activity | Mn⁺⁺ Relative |
| pH | (%) | Activity (%) |
| 3 | 0 | 0 |
| 4 | 50 | 105.8 |
| 5 | 100 | 173.1 |
| 6 | 119.2 | 176.9 |
| 7 | 103.8 | 165.4 |
| 8 | 46.2 | 74 |

These results show that the Mn⁺⁺ induced enhancement of the hydrolysis of cooked dent corn starch by the β-amylase from barley is the greatest at a pH range of 5.0-6.0.

What is claimed is:

1. A method of hydrolyzing a starch substrate comprising:
contacting the starch substrate with one or more amylase family enzymes in the presence of greater than 0.001 mM manganese ion, provided that the one or more amylase family enzymes is not Neurospora crassa glucoamylase or Lentinula edodes glucoamylase.

2. A method for increasing the enzymatic rate of hydrolysis of a starch substrate comprising:
a) providing a reaction mixture comprising at least one starch substrate, one or more amylase family enzymes, provided that the one or more amylase family enzymes is not Neurospora crassa glucoamylase or Lentinula edodes glucoamylase, and greater that 0.001 mM manganese ion; and
b) incubating the reaction mixture under conditions that allow the one or more amylase family enzymes to react with the starch substrate.

3. The method of claim 2, wherein the conditions allow the one or more amylase family enzymes to catalyze the hydrolysis of at least 1% of the α-1,4 bonds, the α-1,6 bonds, or both the α-1,4 bonds and the α-1,6 bonds in the starch substrate.

4. The method of claim 1, wherein the reaction comprises a buffer and has a pH of less than 8.

5. The method of claim 4, wherein the pH of the reaction mixture is from 4 to 7.

6. The method of claim 1, wherein the starch substrate is a naturally-occurring starch, a modified starch, or an intermediate of starch hydrolysis.

7. The method of claim 6, wherein the starch substrate is a cereal starch, a root and tuber starch, or a legume starch.

8. The method of claim 1, wherein the one or more amylase family enzymes and starch substrate are contacted in the presence of at least 0.01 mM manganese ions.

9. The method of claim 8, wherein the one or more amylase family enzymes and starch substrate are contacted in the presence of from 0.01 mM manganese ions to 100 mM manganese ions.

10. The method of claim 9, wherein the one or more amylase family enzymes and starch substrate are contacted in the presence of 0.1 mM to 10 mM manganese ions.

11. The method of claim 1, wherein the one or more amylase family enzymes and starch substrate are contacted in the presence of greater than 1 mM manganese ion.

12. The method of claim 1, wherein the starch substrate is contacted with one or more α-amylases from the following microorganisms, plants, and animals:
Aeromonas hydrophila
Alteromonas haloplanktis
Dictyoglomus thermophilum
Escherichia coli
Bacillus amyloliquefaciens
Bacillus megaterium
Bacillus sp. (strain B1018)
Bacillus circulans
Bacillus stearothermophilus
Bacillus licheniformis
Bacillus subtilis
Paenibacillus polymyxa (Bacillus polyinyxa)
Butyrivibrio fibrisolvens
Methanococcus jannaschii
Streptoniyces lividans
Streptomyces violaceus (Streptomyces venezuelae)
Streptomyces griseus
Streptonyces limosus (Streptomyces albidoflavus)
Streptomyces hygroscopicus
Streptomyces thermoviolaceus
Colstridium acetobutylicum
Thermoanaerobacter thermosulfurogenes (Clostridium thermosulfurogenes)
Thermoanaerobacter ethanolicus (Clostridium thermohydrosulfuricum)
Tiermoanaerobacter thermohydrosulfuricus (Clostridium thermohydrosulfuricum)
Therinoanaerobacter saccharolyticum
Thermomonospora curvata
Pyrococcus furiosus
Pyrococcus horikoshii
Salmonalla typhimurium
Aspergillus niger
Aspergillus awamori
Aspergillus oryzae
Aspergillus shirousani
Schizosaccharomyces pombe (Fission yeast)
Saccharomycopsis fibuligera (Yeast)
Debaryomyces occidentalis (Yeast) (Schwannioinyces occidentalis)
Oryza sativa (Rice)
Triticuin aestivum (Wheat)
Hordeum vulgar (Barley)
Vigna mungo (Rice bean) (Black grain)
Drosophila melanogaster (Fruit fly)
Drosophila mauritiana
Drosophila yakuba
Aedes aegypti (Yellowfever mosquito)
Dermatophagoides pteronyssinus (House-dust mite)
Tribolium castaneum (Redflour beetle)
Pecten maximus (King scallop) (Pilgrim's clam)
Tenebrio molitor (Yellow mealworm)
Porcine Pancreas (Pig), Homo sapiens (Human)
Rattus norvegicus (Rat)
Mus muscluas (Mouse).

13. The method of claim 1, wherein the starch substrate is contacted with one or more amylases from the following microorganisms and plants:
Arabidopsis thaliana (Mouse-ear cress)
Bacillus firmus
Zea mays (Maize)
Secale cereale (Rye)
Trifolium repens (Creeping white clover)
Bacillus cereus
Hordeum vulgare (Barley)
Medicago sativa (Alfalfa)
Glycine max (Soybean)
Vigna unguiculata (Cowpea)
Bacillus circulans
Ipomoea batatas (Sweet potato)
Paenibacillus polymyxa (Bacillus polymyxa)
Thermoanaerobacter thermosulfurogenes (Clostridium thermosulfurogenes)
Triticum aestivuin (Wheat).

14. The method of claim 1, wherein the starch substrate is contacted with one or more glucoamylases from the following microorganisms and plants
Arxula adeninivorans (Yeast),
Aspergillus niger
Candida albicans (Yeast)
Hormoconis resinae (Creosote fungus)
Saccaromycopsis fibuligera (Yeast)
Saccharomyces diastaticus (Yeast)
Maltase-glucoamylase, intestinal
Aspergillus awamori
Aspergillus oryzae
Clostridium sp. (strain G0005)
Schizosaccharomyces pombe (Fission yeast)
Sacchormycopsis fibuligera (Yeast)
Aspergillus kawachi (Aspergillus awamor var. kawachi)
Aspergillus shrousaini
Debaryomyces occidentalis (Yeast) (Schwannioinyces occidentalis)
Rhizopus oryzae (Rhizopus delemar)
Saccharomyces cerevisiae (Baker's yeast)
Saccharomyces diastaticus (Yeast).

15. The method of claim 14, wherein the reaction mixture comprises greater than 1.0 mM manganese ion.

16. The method of claim 1, wherein the reaction mixture comprises one or more of the following amylase family enzymes:
the glucoamylase from Rhizopus Sp, the glucoamylase from Aspergillus niger, the α-amylase from Aspergillus oryzae, the α-amylase from porcine pancreas, the α-amylase from Bacillus amyloliquefaciens, the α-amylase from Bacillus licheniformis, the α-amylase from Bacillus subtilis, the α-amylase from human saliva, the industrial α-amylase from Novozymes known as Termamyl, the β-amylase from barley and the β-amylase from sweet potato.

17. The method of claim 16, wherein the reaction mixture comprises more than 1 mM manganese ion.

18. The method of claim 1, wherein the manganese ion is provided in the form of one of the following manganese salts:
manganese chloride, manganese acetate, manganese sulfate, manganese bromide, manganese difluoride, manganese nitrate, manganese oxalate, manganese benzoate, manganese phosphate and manganese phosphate dibasic.

19. The method of claim 1, wherein the one or more amylase family enzymes and starch substrate are contacted in the presence of the manganese ion and more than 0.001 mM calcium ions.

20. The method of claim 1, wherein the starch substrate is contacted with at least one α-amylase and at least one β-amylase enzyme.

21. The method of claim 1, wherein the starch substrate is contacted with at least one α amylase and at least one glucoamylase enzyme.

22. The method of claim 1, wherein the starch substrate is contacted with at least one β-amylase enzyme and at least one glucoamylase enzyme.

23. The method of claim 1, wherein the starch substrate is contacted with at least one α-amylase, at least one β-amylase enzyme, and at least one glucoamylase enzyme.

24. A method for increasing the enzymatic rate of hydrolysis of a starch substrate by Lentiluna edodes glucoamylase comprising:
a) providing a reaction mixture comprising at least one starch substrate, Lentiluna edodes glucoamylase, and greater that 0.001 mM manganese ion; and
b) incubating the reaction mixture under conditions that allow the Lentiluna edodes glucoamylase to react with the starch substrate.

25. A method of hydrolyzing a starch substrate comprising:
contacting the starch substrate with greater than 0.001 mM calcium ion, a β-amylase enzyme, a glucoamylase enzyme provided that the glucoamylase is not the glucoamylase from Lentiluna edodes, the glucoamylase from Neurospora crassa, the glucoamylase from Aspergillus terreus, or the glucoamylase from Aspergillus satoi, or both a β-amylase enzyme and a glucoamylase enzyme.

26. A method for increasing the enzymatic rate of hydrolysis of a starch comprising:
a) providing a reaction mixture comprising at least one starch substrate, greater than 0.001 mM calcium ion, and one or more or any combination of the following enzymes: β-amylase and glucoamylase, provided that the glucoamylase is not from Lentiluna edodes, Neurospora crassa, Aspergillus terreus, or Aspergillus satoi; and
b) incubating the reaction mixture under conditions that allow the one or more or any combination of enzymes to react with the starch substrate.

27. The method of claim 26, wherein the conditions allow the one or more or any combination of the enzymes to catalyze the hydrolysis of at least 1% of the α-1,4 bonds, the α-1,6 bonds, or both the α-1,4 bonds and the α-1,6 bonds in the starch substrate.

28. The method of claim 26, wherein the reaction comprises a buffer and has a pH of less than 8.

29. The method of claim 26, wherein the pH of the reaction mixture is from 4 to 7.

30. The method of claim 26, wherein the starch substrate is a naturally-occurring starch molecule, a modified starch substrate, or an intermediate of starch hydrolysis.

31. The method of claim 26, wherein the starch substrate is a cereal starch, a root and tuber starch, or a legume starch.

32. The method of claim 26, wherein the one or more or any combination of enzymes and starch substrate are contacted in the presence of at least 0.01 nM calcium ions.

33. The method of claim 26, wherein the one or more or any combination of enzymes and starch substrate are contacted in the presence of 0.1 mM to 100 mM calcium ions.

34. The method of claim 26, wherein the one or more or any combination of enzymes and starch substrate are contacted in the presence of greater than 1.0 mM calcium ions.

35. A method of hydrolyzing a starch substrate comprising:
contacting the starch substrate with β-amylase in the presence of greater than 0.001 mM manganese ion, calcium ion, magnesium ion, strontium ion, barium ion or any combination of said ions.

36. A method of hydrolyzing a starch substrate comprising:
contacting the starch substrate with a glucoamylase enzyme in the presence of greater than 0.001 mM manganese ion, calcium ion, lithium ion, potassium ion, or any combination of said ions, provided that the glucoamylase is not from Lentiluna edodes, Neurospora crassa, Aspergillus terreus, or Aspergillus satoi.

37. A composition comprising greater than 1.0 mM manganese ion and one or more or any combination of the following enzymes: an α-amylase, a β-amylase and a glucoamylase, provided that the glucoamylase is not the glucoamylase from Neurospora crassa or the glucoamylase from Lentiluma edodes.

38. The composition of claim 37, wherein the one or more or any combination of enzymes are isolated.

39. The composition of claim 37, wherein the composition comprises a buffer and has a pH of less than 8.

40. The composition of claim 37, wherein the composition comprises greater than 0.001 mM calcium ions.

41. A composition comprising manganese ion and one or more or any combination of the following enzymes: solid α-amylase, solid β-amylase and solid glucoamylase.

42. A kit comprising:
manganese ion;
one or more or any combination of the following enzymes: an α-amylase, a β-amylase and a glucoamylase, provided that the glucoamylase is not the glucoamylase from Neurospora crassa or the glucoamylase from Lentiluma edodes, and
instructions for using the one or more or any combination of enzymes and the manganese ion to hydrolyze a starch substrate,
wherein the one or more or any combination of the enzymes and manganese ion are in one or more containers.

43. The kit of claim 42, wherein the one or more or any combination of enzymes are isolated.

44. A kit comprising manganese ion and one or more or any combination of the following enzymes: solid α-amylase, solid β-amylase and solid glucoamylase, wherein the one or more or any combination of the enzymes and manganese ion are in one or more containers.

45. The kit of claim 44, further comprising instructions for using the one or more or any combination of enzymes and the manganese ion to hydrolyze a starch substrate.

46. A kit comprising:
one or more or any combination of the following ions: calcium ion, magnesium ion, barium ion, and strontium ion;
one or more or β-amylase enzymes; and
instructions for using the one or more or any combination of said ions and the one or more β-amylase enzymes to hydrolyze a starch substrate,
wherein the one or more or any combination of said ions and the one or more β-amylase enzymes are in one or more containers.

47. The kit of claim 46, wherein the one or more β-amylase enzymes are isolated.

48. A kit comprising one or more or any combination of the following ions: calcium ion, magnesium ion, barium ion, and strontium ion, and one or more solid β-amylase enzymes; wherein the one or more or any combination of said ions and the one or more solid β-amylase enzymes are in one or more containers.

49. The kit of claim 48, further comprising instructions for using the one or more β-amylase enzymes and one or more or any combination of said ions to hydrolyze a starch substrate.

50. A kit comprising:
one or more or any combination of the following ions: calcium ion, potassium ion, and lithium ion;
one or more glucoamylase enzymes, provided that none of the one or more glucoamylase enzymes is from Lentiluna edodes, Neurospora crassa, Aspergillus terreus, or Aspergillus satoi; and
instructions for using the one or more or any combination of said ions and the one or more glucoamylase enzymes to hydrolyze a starch substrate,
wherein the one or more or any combination of said ions and the one or more glucoamylase enzymes are in one or more containers.

51. The kit of claim 50, wherein the one or more glucoamylase enzymes are isolated.

52. A kit comprising one or more or any combination of the following ions: calcium ion, lithium ion, and potassium ion and one or more solid glucoamylase enzymes, provided that none of the one or more solid glucoamylase enzymes is from Lentiluna edodes, Neurospora crassa, Aspergillus terreus, or Aspergillus satoi;
wherein the one or more or any combination of said ions and the one or more solid glucoamylase enzymes are in one or more containers.

53. The kit of claim 52, further comprising instructions for using the one or more glucoamylase enzymes and the one or more or any combination of said ions to hydrolyze a starch substrate.
